(19) United States
(12) Patent Application Publication
(10) Pub. No.: US 2022/0082546 A1
(43) Pub. Date: Mar. 17, 2022

(54) MULTI-LOCATION TIME-DIVISION WATER QUALITY MONITORING SYSTEM

(71) Applicant: Shang-Jung WU, Taoyuan City (TW)

(72) Inventor: Shang-Jung WU, Taoyuan City (TW)

(21) Appl. No.: 17/019,905

(22) Filed: Sep. 14, 2020

(51) Int. Cl. G01N 33/18

(52) U.S. Cl. CPC G01N 33/1826; G01N 33/1893

(57) ABSTRACT

A multi-location time-division water quality monitoring system includes a water quality sensing apparatus and a management station. An unmanned vehicle of the water quality sensing apparatus can receive a task instruction from a controlled module through a wireless transmission module, and can fly to and from sensing location in cooperation with the positioning module and based on preset flight paths. After reaching the sensing location, the unmanned vehicle executes a tack of collecting water quality parameter data through the water quality sensing module, and transmits the water quality parameter data to the management station or a background server. The management station can automatically control a washing machine to clean the water quality sensing module, and control a power supplement apparatus to supplement the unmanned vehicle with power, so that an on-site operator can use single water quality sensing apparatus to monitor multiple sensing locations, to monitoring wide environment.

### BACKGROUND OF THE INVENTION

### 1. Field of the Invention

The present invention provides a multi-location time-division water quality monitoring system, and more particularly to a system combining a water quality sensing apparatus which includes an unmanned vehicle mounted with a water quality sensing module, and a management station which can automatically charge the unmanned vehicle and clean a water quality sensing module, so as to form a long-distance unmanned water quality monitoring system for monitoring changes in water qualities of multiple sensing locations in a interval time.

### 2. Description of the Related Art

In recent years, natural seawater, aquaculture ponds or aquariums are used for stocking useful aquatic animals or products, such as fish, shrimp or aquatic plants and the likes. Besides breeding pond apparatus, other factors such as water quality, stocking amount, stocking time, feeding amount, temperature of the breeding pond are all important factors affecting the breeding result. In order to provide the breeding aquatic products with better growth conditions, most of the breeding ponds are currently equipped with breeding machines, such as waterwheels, automatic feeders, filters, and a variety of electronic sensing apparatuses are also used to obtain water quality information in the breeding pond, so that changes of the water quality of the breeding pond can be sensed and returned to the farmer to take corresponding actions in real time, thereby preventing from death of the breeding aquatic animals due to the setting of the water quality conditions and the ecological environment not meeting requirement and change in the growth of aquatic animals.

However, generally, organisms in the breeding pond may come from different environments and require different water quality conditions, and it means that the ecological composition in the breeding pond is quite complicated. Therefore, the water quality conditions (such as dissolved oxygen, temperature, PH, salinity, etc.), the degree of water pollution (such as ammonia nitrogen, suspended solids, oxidation reduction potential, etc.), zooplankton and phytoplankton, are the keys to prevent disease of aquatic products and to have good breeding result, and good water quality is very important for the health of aquatic animals. The common method of maintaining good water quality is to monitor the PH value and dissolved oxygen of water in the breeding pond, and if the PH value of the water deviates from the allowable value, a pump is started to pump and exchange water, or some necessary stabilizers such as lime, humic acid, and probiotics can be added into the water to disinfect and decompose the spoilage substances at the bottom of the breeding pond, further provide algae with nutrients and adjust the PH value of the water quality, and maintain the density of zooplankton and phytoplankton. Later, the water quality is tested every period of time until the PH value is within the allowable range. When the dissolved oxygen in the water is insufficient, a waterwheel is started to hit water or an aerator is started to supplement dissolved oxygen in the water, so as to ensure the balance and continuation of the ecology in the breeding pond.

In a conventional continuous water quality monitoring system, each breeding pond in a different area needs a water quality collection apparatus for collecting data; however, in order to centralize and unify controls of multiple different breeding ponds and monitor relevant data of water quality parameters (such as water temperature, dissolved oxygen, PH value, salinity, ammonia nitrogen, suspended solids, redox potential, etc.), multiple water quality collection apparatuses are required, and it causes low willingness of farmers to adopt the continuous water quality monitoring system.

As shown in FIG. 2, which is a block schematic diagram of a conventional multi-location water quality monitoring system. In the multi-location water quality monitoring system, a plurality of sensing modules A1 (including a water quality sensor and a signal processing unit) of water quality collection apparatuses A are disposed in different breeding ponds, respectively, and a signal collection and storage unit A2 can collect and store the water quality parameter data of the water body collected by the water quality sensor through wired or wireless transmission, and then upload the collected data to the a background server or a cloud processing platform B through internet, to perform various monitoring or control operations. However, the water quality sensor of the multi-location water quality monitoring system is usually in the water body of the breeding pond for a long time, if the detected water body is highly polluted or is rich in zooplankton and phytoplankton, algae and shell organisms are easily attached on the optical water quality sensor, and it results in rapid attenuation or distortion of the sensing signal; furthermore, the electrochemical water quality sensor is also easy to rapidly age because of continuous reaction of the electrode thereof, so personnel must frequently reach the breeding ponds at different locations for calibration, repair and maintenance. Therefore, the dependence of the conventional multi-location water quality monitoring system on manpower is extremely high, and once the above-mentioned maintenance is neglected, it will cause a significant impact and even lead to system failure and misjudgment.

Therefore, in the era in which the water qualities of breeding ponds are automatically monitored and controlled, various water quality parameters in closed water bodies do not change frequently or violently, so how to develop a multi-location and time-division water quality monitoring and control system, which can reduce labor cost in the maintenance of the water quality sensor, effectively solve many inconveniences and problems, reduce cost in unified management of multiple breeding ponds and maintain the water quality in the best state, is an important issue in the industry.

### SUMMARY OF THE INVENTION

In order to solve the conventional problems, the inventors develop a multi-location time-division water quality monitoring system according to collected data, evaluations, and multiple tests and modifications, and years of experience in the industry.

An objective of the present invention is that an unmanned vehicle of a water quality sensing apparatus is mounted with a water quality sensing module, and the water quality sensing apparatus is in cooperation with a management station, which includes a washing machine and a power supplement apparatus, to form a long-distance unmanned water quality monitoring system; the water quality sensing apparatus can receive a task instruction, and automatically fly to reach the specified sensing location based on at least two preset flight paths, to execute various tasks of collecting water quality parameter data, and then transmit back and store the water quality parameter data to one of a management station and a background server; after the water quality sensing apparatus flies back to the management station, the management station can automatically complete the operations of supplementing the unmanned vehicle with power and cleaning the water quality sensing module, so that the on-site operator can use single water quality sensing apparatus to monitor changes in water qualities of a plurality of sensing locations in real time, and understand the status of water in the currently-sensing location based on a monitoring result, to achieve the purpose of monitoring wide environment by a multi-location and time-division manner; furthermore, it also does not need to setup multiple water quality collection devices for different water bodies to be monitored, so that implementation costs can be reduced more effectively.

Another objective of the present invention is that with configuration of the single water quality sensing apparatus in cooperation with the management station, the water quality parameter data of the multiple sensing locations can be collected by using one water quality sensing module mounted on the single water quality sensing apparatus, and the water quality parameter data can be transmitted back to and stored in one of the management station or the background server; the user can just issue a task instruction to automatic route or increase the flight path and setting of the flight path through a management program of the background server or the cloud processing platform, so as to increase amount of the sensing locations without additional expense, and the on-site operator can monitor the changes in water qualities of the multiple sensing locations in real time, to perform corresponding action according to the monitoring results.

Another objective of the present invention is that the unmanned vehicle of the water quality sensing apparatus can receive the task instruction issued from the management program of the background server or the cloud processing platform through the wireless transmission module, and when it determines that the weather condition is suitable for flying based on the weather information, which is provided by the management program and includes rainfall and wind speed, all online water quality sensing apparatuses can automatically execute collection tasks, and the management program can be linked to the open data platform of the meteorological administration to obtain the weather information of different areas through an application programming interface, so as to plan and correct the latest flight path based on the real-time weather condition.

Another objective of the present invention is that when the unmanned vehicle flies to the sensing location, the controlled module can perform positioning of the unmanned vehicle to guide the unmanned vehicle to accurately fly to the specified position and height according to a positioning label disposed at the sensing location, so as to prevent the unmanned vehicle from being affected, by terrain, vegetation and trees surrounding the specified location, to make wrong flight decision.

### BRIEF DESCRIPTION OF THE DRAWINGS

The structure, operating principle and effects of the present invention will be described in detail by way of various embodiments which are illustrated in the accompanying drawings.

FIG. 1 is a block diagram of a preferred embodiment of a multi-location time-division water quality monitoring system of the present invention.

FIG. 2 is a block diagram of a conventional multi-location water quality monitoring system.

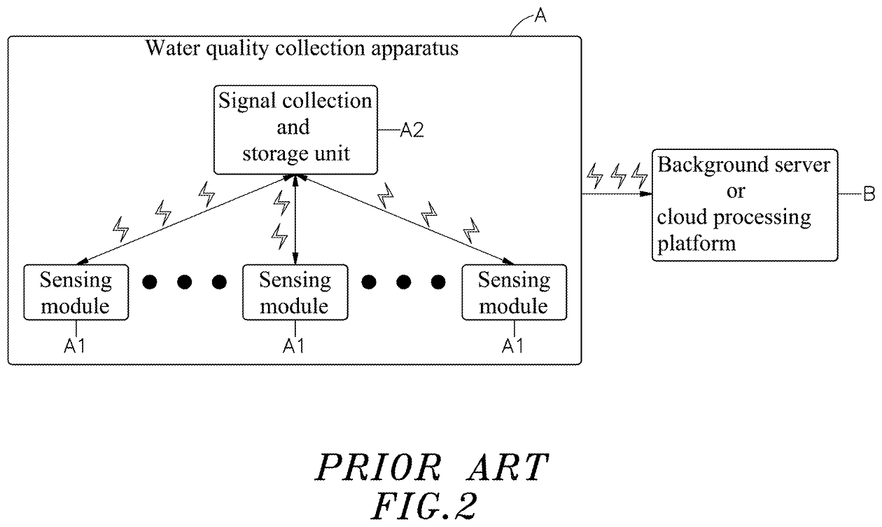

### DETAILED DESCRIPTION OF THE PREFERRED EMBODIMENTS

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that, although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on, connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise” and variations such as “comprises” or “comprising”, will be acknowledged to imply the inclusion of stated elements but not the exclusion of any other elements.

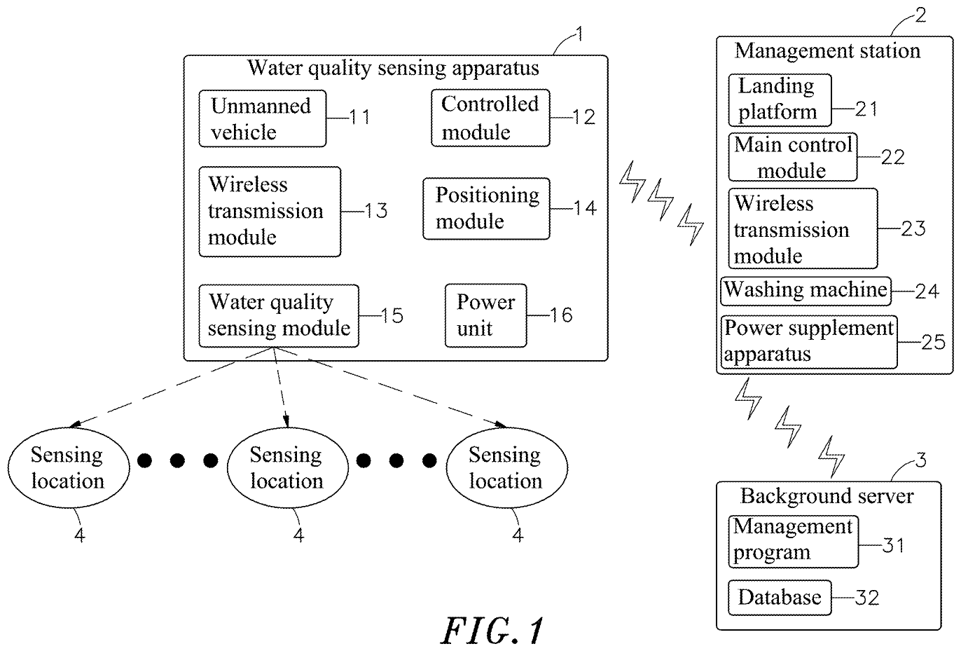

Please refer to FIG. 1, which is a block diagram of a preferred embodiment of a multi-location time-division water quality monitoring system of the present invention. As shown in FIG. 1, the multi-location time-division water quality monitoring system includes at least one water quality sensing apparatus 1 and a management station 2.

The water quality sensing apparatus 1 includes an unmanned vehicle 11, a controlled module 12, a wireless transmission module 13, a positioning module 14, a water quality sensing module 15, and a power unit 16. Preferably, the unmanned vehicle 11 can be an unmanned aerial vehicle or an unmanned aircraft system (UAS), and the unmanned vehicle 11 is also called as UAV for aerial photography, or a remote-controlled UAV. A power device is mounted on a vehicle frame of the unmanned vehicle 11, and the power device mainly includes paddles and a motor, to form the unmanned vehicle 11 as a rotorcraft, a fixed-wing aircraft or a helicopter rotor aircraft, and the power device can drive vehicle body to fly through the reaction force generated while the paddles are rotating.

The unmanned vehicle 11 generally use a sensor, such as a gyroscope, an accelerometer, a magnetometer or a barometer, for feedback control, and use the controlled module 12 to receive the task instruction or the control signal through the wireless transmission module 13. The controlled module 12 can include a controller, a signal processing unit and a storage unit, and can be in cooperation with the positioning module 14 to obtain information about the posture, heading or height of the unmanned vehicle 11, so as to stably control the unmanned vehicle 11 to fly at the specified position and height for positioning and navigation, and track and record flight trace and movement of the unmanned vehicle 11 in real time, so as to perform the functions of stabilizing and controlling the posture and managing the task apparatus, and emergency control function. The positioning module 14 can include global positioning system (GPS) or inertial navigation system.

In this embodiment, the unmanned vehicle 11 includes the water quality sensing module 15 mounted on a lower part of the vehicle frame thereof, the water quality sensing module 15 includes at least one water quality sensor configured to sense physical parameters including, but not limited to, water temperature, water pressure, etc.; chemical parameters such as dissolved oxygen, PH value, salinity, ammonia nitrogen (NH3-N), suspended solids, oxidation reduction potential; or other water quality parameters, or sense a combination of any one or more of the above water quality parameters. After the controlled module 12 integrates the collected data of the water quality parameters, the wireless transmission module 13 can return and store the integrated data to a database 32 of the management station 2 or a background server 3 through 3G, 4G-LTE or 5G-NR mobile communication network. Furthermore, the unmanned vehicle 11 can include the power unit 16 mounted in the vehicle body and configured to be charged or exchanged. Preferably, the power unit 16 can be a lithium polymer battery, solar battery or fuel battery, to provide the overall required power of the water quality sensing apparatus 1.

Furthermore, the controlled module 12 of the water quality sensing apparatus 1 can obtain the task instruction issued by a management program 31 of the background server 3 or the cloud processing platform through the wireless transmission module 13, and automatically plan at least two flight paths between two fixed locations. When it is determined that weather condition is suitable for flying based on the weather information provided by the management program 31, all online water quality sensing apparatuses 1 can automatically complete the collection tasks based on the issued task instructions, and quickly transmit images captured by the water quality sensing apparatus 1 and the collected water quality sensing data in real time, so that an on-site operator can understand task execution status of the water quality sensing apparatus 1, and also can stream video and audio of the unmanned vehicle, set the flight area and verify the flight rights in real time, so as to operate the flight action. The management program 31 of the background server 3 can be linked to an open data platform of the meteorological administration to obtain the weather information of different areas through the application programming interface, to plan and correct the latest flight path based on the real-time weather condition. The weather information includes rainfall and wind speed.

Furthermore, the unmanned vehicle 11 can include a camera module mounted at a front part of the vehicle frame thereof and configured to transmit the captured images and the GPS coordinates of the positioning module 14 back to the background server 3 through the wireless transmission module 13 in real time, so as to enable the on-site operator to monitor the current task execution status and set the flight path through the image displayed on the screen. However, it should be noted that the manner of receiving the task instruction through the controlled module 12, and enabling the unmanned vehicle 11 to automatically complete the task in cooperation with the positioning module 14 and based on the set flight path, and the manner of collecting water through the water quality sensing module 15 are well known in the art, and the details of the above-mentioned manners are not key features of the present invention, so the details are described in the following paragraphs together.

The management station 2 includes a landing platform 21, a main control module 22, a wireless transmission module 23, a washing machine 24 and a power supplement apparatus 25. The management stations 2 can be a fixed or mobile base station. A platform release frame can be mounted on the landing platform 21, so that the main control module 22 can control the platform release frame to hold the unmanned vehicle 11 firmly before the unmanned vehicle 11 is released, and when the unmanned vehicle 11 is to release, the platform release frame can assist the unmanned vehicle 11 to lift off smoothly; however, the present invention is not limited to this example. The management station 2 can be used to manage all online water quality sensing apparatuses 1, to make sure whether each unmanned vehicle 11 is operating normally; when detecting an abnormal condition of the unmanned vehicle 11, the management station 2 can issue a warning signal to the on-site operator for immediate response, and the related details are described in the following paragraphs together.

In this embodiment, the washing machine 24 can include a cleaning sink, an inlet pipe and a drain pipe which are connected to the cleaning sink, and the cleaning sink is set with an electric brush or a nozzle disposed therein. When the water quality sensing apparatus 1 is positioned on the landing platform 21, a sensor head of the water quality sensing module 15 can be inserted into the cleaning sink, and the electric brush or the nozzle can wash, clean or brush the sensor head, and the water or cleaning liquid for the cleaning operation can be discharged through the drain pipe, so as to implement the functions of automatic cleaning (brushing), water inlet, drainage, and agent (such as cleaning agent) addition.

The power supplement manner adopted by the power supplement apparatus 25 to supplement power to the water quality sensing apparatus 1 clamped on the landing platform 21 can include, but not limited to, a charging manner, a battery exchange manner, or a combination of charging manner and the battery exchange manner. In this embodiment, the power supplement apparatus 25 can use the charging unit on the platform to charge the lithium polymer battery of the power unit 16 by a wired or wireless power transmission manner, and the power supplement apparatus 25 can automatically stop charging after the battery is charged fully or charged for a preset time, so that the water quality sensing apparatus 1 can have enough power to fly between the management station 2 and the sensing location without limitation of the distance and flight time. In an embodiment, a solar panel can be used as a power source. In the battery exchange manner, a robot arm is used to remove the battery in the battery bay of the unmanned vehicle 11 first, and place the battery into the charger of the platform for fast charging, and then grab and insert a fully charged battery into the battery bay, so as to quickly complete the battery exchange. Next, the water quality sensing apparatus 1 is restarted to continue to the remaining various tasks.

When the system of the present invention is in use, the controlled module 12 of the water quality sensing apparatus 1 obtains the task instruction issued by the management program 31 of the background server 3 or the cloud processing platform through the wireless transmission module 13, to enable the unmanned vehicle 11 to fly to and from the sensing location 4 in cooperation with the positioning module 14 and based on at least two flight paths, which are automatically planned and set. After the unmanned vehicle 11 successfully takes off from the landing platform 21 of the management station 2, the unmanned vehicle 11 can automatically fly to the specified sensing location 4 to execute various collection tasks. Preferably, the sensing location 4 can be, but not limited to, a breeding pond or fish farm, or a natural water area (such as rivers, lakes, etc.), or an artificial water body (such as pond, barrage, reservoir, etc.). At least one positioning label can be disposed at each sensing location 4 to provide foolproof positioning for the unmanned vehicle 11. When the unmanned vehicle 11 flies to the sensing location 4, the controlled module 12 can first recognize the positioning label (such as a label or a pattern) through the camera module, and then position the unmanned vehicle 11 and calculate a predetermined descended position and height based on the positioning label, to guide the unmanned vehicle 11 to accurately descend to above the water surface in sensing location 4 by a distance, so as to prevent the unmanned vehicle 11 from being affected, by terrain, vegetation and trees surrounding the specified location, to make wrong flight decision; furthermore, the above-mentioned operation also make the unmanned vehicle 11 hover at a predetermined height to facilitate the execution of various tasks.

When the unmanned vehicle 11 reaches above the water surface at the specified sensing location 4, the retractable mechanism of the unmanned vehicle 11 can drive the water quality sensing module 15 to swing downwardly to change the direction and angle of the sensor head of the water quality sensor; or the unmanned vehicle 11 can be directly parked on the water surface through an internal buoyancy chamber or a floating kit additionally mounted, and the sensor head of the water quality sensing apparatus can then be extended under the water surface to collect the water quality parameter data in the water body. After the controlled module 12 integrates the collected water quality parameter data, the wireless transmission modules 13 and 23 can transmit back and store the integrated data to the main control module 22 of the management station 2 or the database 32 of the background server 3, so that the background server 3 or the cloud processing platform can perform various monitoring or control operation, and the multiple different sensing locations 4 to be monitored can be flexibly changed. Therefore, the on-site operator can use single water quality sensing apparatus 1 to monitor multiple sensing locations 4, thereby achieving the purpose of monitoring wide environment by a multi-location and time-division manner; furthermore, it does not need to setup multiple water quality collection apparatuses for different water bodies to be monitored, so the implementation costs can be effectively reduced.

After the unmanned vehicle 11 returns to the management station 2 and lands on the landing platform 21, and is firmly clamped by the platform release frame, the main control module 22 automatically controls the washing machine 24 to clean the sensor head of the water quality sensing module 15, and control the power supplement apparatus 25 to quickly supplement the power unit 16 with power; and after being cleaned and supplemented with power completely, the unmanned vehicle 11 continues to fly to the next sensing location 4 for task of collecting water quality parameter data, based on the set flight path, and the above-mentioned operations are repeated until all tasks are completed. However, in actual application, the present invention is not limited to above-mentioned embodiment. For example, the unmanned vehicle 11 can first complete more than one according to the flight distance and time supported by the power unit 16, and then return to the management station 2 for cleaning and power supplement, and the main control module 22 can track and record the flight trajectory and movement of the unmanned vehicle 11 in real time, so that the background server 3 or the cloud processing platform can plan the next flight path, and the maintenance of the water quality sensing module 15 can be implemented by automatic cleaning operation of the washing machine 24, thereby reducing required manpower and maintenance costs.

Therefore, the main inventive concept of the present invention is that the unmanned vehicle 11 of the water quality sensing apparatus 1 is mounted with the water quality sensing module 15; and the water quality sensing apparatus 1 and the management station 2, which includes the washing machine 24 and the power supplement apparatus 25, can form a long-distance unmanned water quality monitoring system, the water quality sensing apparatus 1 can receive a task instruction, and automatically fly to and from the specified sensing location 4 based on the at least two preset flight paths, to execute various tasks of collecting water quality parameter data, and after the water quality sensing apparatus 1 returns to the management station 2 and is automatically cleaned and supplemented with power completely, the water quality sensing apparatus 1 can continue to fly to the next sensing location 4 based on the set flight path to execute the task of collecting the water quality parameter data until all tasks are completed.

With the configuration of the single water quality sensing apparatus 1 in cooperation with the management station 2, the water quality parameter data of the multiple sensing locations 4 can be collected by using only one water quality sensing module 15 mounted on the single water quality sensing apparatus 1, and the water quality parameter data is transmitted back to and stored in the management station 2 or the background server 3; furthermore, the user can just use the task instruction issued by the management program 31 or the cloud processing platform of the background server 3 to automatically plan or increase the setting and arrangement of the flight path, so that the sensing location 4 can be increased without additional cost, and the flight path can also be planned and modified based on real-time weather condition; as a result, the on-site operator can monitor the changes in water quality of the multiple sensing locations 4 in real time, and understand the current statuses of waters to be monitored according to the monitoring results, and take corresponding action such as the action of automatically controlling pumps for water changes, controlling a waterwheel to draw water, controlling a water temperature heater or chillers to adjust water temperature, or controlling an automatic feeder to spray feed.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A multi-location time-division water quality monitoring system, comprising at least one water quality sensing apparatus and a management station, wherein the water quality sensing apparatus comprises an unmanned vehicle, a controlled module, a wireless transmission module, a positioning module, a water quality sensing module and a power unit, and the water quality sensing module and the power unit are mounted on the unmanned vehicle, the controlled module receives a task instruction through the wireless transmission module and is configured to operate the unmanned vehicle to move to and from sensing locations in corporation with the positioning module and based on at least two preset flight paths, wherein when the unmanned vehicle flies to reach the sensing location, the unmanned vehicle executes a task of collecting water quality parameter data through the water quality sensing module, and transmits back the water quality parameter data to the management station;
wherein the management station comprises a landing platform, a main control module, a wireless transmission module, a washing machine, and a power supplement apparatus, the landing platform is configured to fasten the unmanned vehicle, the main control module is configured to manage an operation of the water quality sensing apparatus through the wireless transmission module, and receive the water quality parameter data, and after the unmanned vehicle flies back to the landing platform from the sensing location, the main control module automatically controls the washing machine to clean the water quality sensing module, and controls the power supplement apparatus to supplement power unit with power.

2. The multi-location time-division water quality monitoring system according to claim 1, wherein the unmanned vehicle of the water quality sensing apparatus is one of an unmanned aerial vehicle and an unmanned aircraft system, and the water quality sensing module mounted on the unmanned vehicle comprises at least one water quality sensing configured to sense the water quality parameter data of water bodies at the sensing locations.

3. The multi-location time-division water quality monitoring system according to claim 2, wherein the water quality parameter data comprises any one or a combination of two or more of water temperature, dissolved oxygen, acidity, salinity, ammonia nitrogen (NH3-N), suspended solids, and redox potential.

4. The multi-location time-division water quality monitoring system according to claim 1, wherein the positioning module of the water quality sensing apparatus is one of a global positioning system (GPS) and an inertial navigation system.

5. The multi-location time-division water quality monitoring system according to claim 1, wherein the power unit of the water quality sensing apparatus is a battery, and the power supplement apparatus of the management station supplements the power unit with power by a charging manner or a battery exchange manner.

6. The multi-location time-division water quality monitoring system according to claim 1, wherein the unmanned vehicle of the water quality sensing apparatus obtains the task instruction issued from a management program of one of background server and the cloud processing platform through the wireless transmission module, and automatically plans at least two flight paths between two fixed locations, and executes various collection task when determining that weather condition is suitable for flying based on weather information which is provided by the management program and includes rainfall and wind speed.

7. The multi-location time-division water quality monitoring system according to claim 6, wherein the management program of one of the background server and the cloud processing platform is linked to an open data platform of the meteorological administration to obtain the weather information of multiple regions through the application programming interface.

8. The multi-location time-division water quality monitoring system according to claim 1, wherein the washing machine of the management station include a cleaning sink, the cleaning sink comprising one of an electric brush and a nozzle disposed therein, and configured for insertion of the water quality sensing module.

9. The multi-location time-division water quality monitoring system according to claim 1, wherein a plurality of positioning labels are disposed on the sensing locations, respectively, and each of the plurality of positioning labels is one of a label and a pattern to provide foolproof positioning for the unmanned vehicle.

10. The multi-location time-division water quality monitoring system according to claim 1, wherein each of the plurality of sensing location is one of a breeding pond, a fish farm, natural water area of river, a lake, a pond of artificial water body, a barrage and a reservoir.
